(19) United States
(12) Patent Application Publication
(10) Pub. No.: US 2011/0060040 A1
(43) Pub. Date: Mar. 10, 2011

(54) USES OF ACYLOXYALKYL CARBAMATE PRODRUGS OF TRANEXAMIC ACID

(75) Inventors: Peter A. Virsik, Portola Valley, CA (US); Daniel M. Canafax, Half Moon Bay, CA (US)

(73) Assignee: XenoPort, Inc.

(21) Appl. No.: 12/858,401

(22) Filed: Aug. 17, 2010

Related U.S. Application Data
(60) Provisional application No. 61/240,107, filed on Sep. 4, 2009.

(51) Int. Cl. A61K 31/27; A61P 7/04

(52) U.S. Cl. 514/484

(57) ABSTRACT

Methods of using acyloxyalkyl carbamate prodrugs of trans-4-(aminomethyl)-cyclohexanecarboxylic acid and pharmaceutical compositions thereof are disclosed.

This application claims the benefit under 35 U.S.C. §119(e) of U.S. Provisional Application Ser. No. 61/240,107, filed Sep. 4, 2009, which is incorporated by reference in its entirety.

The disclosure relates to uses of acyloxyalkyl carbamate prodrugs of trans-4-(aminomethyl)-cyclohexanecarboxylic acid and pharmaceutical compositions thereof.

In the surgical setting, skillful surgery combined with blood saving methods and careful management of blood coagulation can reduce unnecessary blood loss and transfusion requirements. Some surgical procedures may be associated with blood loss and/or compromised hemostasis in patients without pre-existing hemostatic abnormalities. Surgical procedures may be associated with hyperfibrinolysis. Typical surgical procedures that may be associated with hyperfibrinolysis include operations requiring cardiopulmonary bypass, orthotopic liver transplantation, and some urological and orthopedic operations. Moreover, there are subgroups of patients who refuse blood transfusion in patients with borderline or mild hemostatic defects such as patients on antiplatelet agents or anticoagulants, patient with hepatic cirrhosis, and those with chronic renal failures. Excessive surgical bleeding causes hypovolemia, hemodynamic instability, anemia and reduced oxygen delivery to tissues with a subsequent increase in postoperative morbidity and mortality. Adverse effects of allogeneic blood transfusion include transmission of infections, diseases, immunosuppression, transfusion-related acute lung injury, transfusion reactions, and graft-vs-host reactions. The cost implication for blood transfusion is also significant and includes the direct blood transfusion costs as well as indirect costs originating from additional treatments and prolonged hospitalization.

Systemic antifibrinolytic agents are widely used in major surgery to prevent fibrinolysis and thus reduce surgical blood loss (Mandy and Webster, Br. J. Anaesthesia 2004. 93(6), 842-58). A recent systematic review of randomized, controlled trials of antifibrinolytic agents in elective surgical patients showed that perioperative administration of antifibrinolytic agents reduced the numbers needing transfusion by one third, reduced the volume needed per transfusion by one unit, and halved the need for further surgery to control bleeding (Henry et al., Cochrane Review, 2004). Tranexamic acid has been shown to be effective in reducing operative bleeding and/or post-operative bleeding in nasal surgery (Yaniv et al., Am J Rhinoplasty 2006, 20(2), 227-229); knee replacement surgery (Zohar et al., Anesth Analg 2004, 99, 1679-83); total knee arthroplasty (Lozano et al., Vox Sanguinis 2008, 95, 39-44; and Cid and Lozano, Transfusion 2005, 45, 1302-1307); hip arthroplasty (Rosencher et al., Transfusion 2003, 43, 459-469); spinal fusion surgery (Wong et al., Anesth Analg 2008, 107, 1479-86); scoliosis surgery (Neilipovitz et al., Anesth Analg 2001, 93, 82-7); complex spine surgery (Colomina et al. Orthopedics 2009, 32(2), 91); orthopedic surgery (Zufferey et al., Anesthesiology 2006, 105, 1034-1046); cardiac surgery (Laupacis et al., Anesth Analg 1997, 85, 1258); orthognathic surgery (Choi et al., J Oral Maxillofac Surg 2009, 67, 125-133); coronary artery bypass surgery (Taghaddomi et al., J Cardiothroacic Vascular Anesthesia 2009, 23(3), 312-5); and prostatic surgery (Dunn and Goa, Drugs 1999, 57, 1005-32). Prophylactic administration of lysine analogs such as tranexamic acid has been shown to reduce post-operative bleeding in cardiopulmonary bypass surgery by 30-40% (Fremes et al., Ann Thorac Surg 1994, 58, 1580-8; and Levi et al., Lancet 1999, 354, 1940-7); reduce total blood loss in patients undergoing total knee arthroplasty by up to 50% and decreased transfusion requirements without increasing the risk of thromboembolic manifestations (Hiippala et al., Anesth Analg 1997, 84(4), 839-44; Jansen et al., Br. J. Anaesth 1999, 83, 596-601; and Veien et al., Acta Anaesthesiol Scand 2002, 46(10), 1206-11); reduce intraoperative blood loss in patients undergoing total hip replacement (Ekback et al., Anesth Analg 2000, 91, 1124-30; and Benoni et al., Acta Orthop Scand 2001, 72, 442-8); and reduce blood loss and transfusion requirements in orthotopic liver transplantation (Boylan et al., Anesthesiolgy 1996, 85, 1043-8; and Dalmau et al., Anesth Analg 2000, 91(1), 29-34).

In general, systemic antifibrinolytics such as tranexamic acid appear to be more effective in reducing bleeding when used prophylactically.

Traumatic hemorrhage is the leading cause of death from wounds in the battlefield and the second leading cause of death in civilian trauma (Kauvar and Wade, Critical Care 2005, 9 (Suppl 5), S1-S9). Responses to trauma and subsequent resuscitation may include hypothermia, hemodilution and acidosis, conditions which can induce coagulopathies in which normal coagulation function is altered and disrupted. Approximately 20% of hemorrhagic deaths are due to compressible wounds (i.e., those that are accessible to direct pressure), treatable with pressure dressings, tourniquets, and mechanical surgical methods. However, the majority (approximately 80%) of hemorrhagic deaths on the battlefield are due to intracavitary hemorrhage, which is not accessible for direct compression such as within the pelvic, abdominal or thoracic cavities (Ryan et al., RTO-MP-FIFM-109 2004). Currently, no method other than surgical intervention can treat intracavitary hemorrhage. In a controlled trial in which antifibrinolytic agents were administered following traumatic injury the results were inconclusive (Coats et al., Cochrane Database Syst Rev 2004, CD004896).

Tranexamic acid (1) (trans-4-(aminomethyl)-cyclohexanecarboxylic acid, Cyklokapron®):

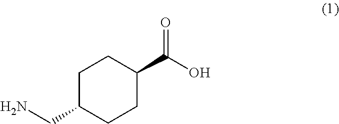

is an antifibrinolytic agent that reversibly blocks lysine binding sites on plasminogen and plasmin, and acts to prevent proteolytic degradation of fibrin clots which form in the normal physiologic process of hemostasis. Both plasminogen and plasmin are activators of fibrinolysis and active clot-lysing agents. Tranexamic acid thus helps to stabilize fibrin clots, which in turn maintains coagulation and helps to control bleeding. Tranexamic acid is usually given as a bolus dose of 10-15 mg/kg intravenously before surgery. In cardiac surgery this can be followed by administration of 1 mg/kg-h for 5-8 hours. However, a wide range of dosage regimens have been used in different surgical procedures (Franck et al., Anesthesiol Clin North America 1999, 17, 799-811). Only a small fraction of administered tranexamic acid is metabolized and most is excreted unchanged by the kidney. Pharmacokinetic studies have revealed that tranexamic acid has a volume of distribution of 9-12 liters and an elimination half-life of about 2 hours.

In prophylactic uses it is desirable to administer tranexamic acid prior to bleeding and is most conveniently accomplished via oral administration. Due to the suboptimal pharmacokinetic properties of tranexamic acid which include modest oral bioavailability (ca. 30%) and a rapid terminal elimination half life of ca. 2 hours, oral formulations such as Cyklokapron® are typically dosed at high concentrations. To address the incomplete gastrointestinal absorption of tranexamic acid prodrug derivatives have been developed (Svahn et al., J. Med. Chem., 1986, 29, 448-453; Svahn et al., EP 0 079 872 B1; Svahn et al., U.S. Pat. No. 4,483,867; Jonsson, WO 94/15904; Svahn et al., Arzneim-Forsch., 1988, 38, 735-738; and Edlund et al., Br. J. Obstet. Gynuecol., 1995, 102, 913-917). The prodrug 1-(ethoxycarbonyl)oxyethyl trans-4-(aminomethyl)-cyclohexanecarboxylate (i.e., Kabi 2161) showed markedly improved oral bioavailability of tranexamic acid in human patients.

Recently, Zerangue et al. described acyloxyalkyl carbamate prodrugs of tranexamic acid that are absorbed from the large intestine and which are appropriate for oral administration using sustained release dosage forms (Zerangue et al., U.S. Pat. No. 7,351,740 and U.S. Published Application No. 2008/0153898). The high oral bioavailability of these acyloxyalkyl carbamate tranexamic acid prodrugs can lead to improved convenience, efficacy, and side effect profile of tranexamic acid therapy.

Acyloxyalkyl carbamate prodrugs of tranexamic acid can be effectively used to reduce or minimize bleeding such as perioperative bleeding and in bleeding due to traumatic injury.

In a first aspect of the present disclosure, a method of treating bleeding caused by a wound in a subject is disclosed, comprising orally administering to the subject a therapeutically effective amount of a tranexamic acid prodrug at least about 1 hour prior to incurring the wound.

A dash (“-”) that is not between two letters or symbols is used to indicate a point of attachment for a substituent. For example, —CONH₂ is attached through the carbon atom.

“Acyl” by itself or as part of another substituent refers to a radical —C(O)R³⁰, where R³⁰ is hydrogen, alkyl, heteroalkyl, cycloalkyl, cycloheteroalkyl, cycloalkylalkyl, cycloheteroalkylalkyl, aryl, heteroaryl, arylalkyl, or heteroarylalkyl, which may be substituted, as defined herein. Examples of acyl groups include, but are not limited to, formyl, acetyl, cyclohexylcarbonyl, cyclohexylmethylcarbonyl, benzoyl, and benzylcarbonyl. In certain embodiments, an acyl group is C₁₋₃ acyl.

“Acylamino” by itself or as part of another substituent refers to a radical —NR³¹C(O)R³², where R³¹ and R³² are independently hydrogen, alkyl, cycloalkyl, cycloheteroalkyl, aryl, arylalkyl, heteroalkyl, heteroaryl, or heteroarylalkyl, which may be substituted as defined herein. Examples of acylamino groups include, but are not limited to, formamido, acetamido, and benzamido.

“1-Acyloxy-alkyl carbamate” “acyloxyalkyl carbamate” refers to an N-1-(acyloxy)alkoxycarbonyl derivative of tranexamic acid as encompassed by compounds of Formula (I) disclosed herein.

“Alkyl” by itself or as part of another substituent refers to a saturated or unsaturated, branched or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.: butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms, in certain embodiments, from 1 to 6 carbon atoms, and in certain embodiments, from 1 to 3 carbon atoms. In certain embodiments, alkyl is C₁₋₆ alkyl, C₁₋₄ alkyl, C₁₋₃ alkyl, methyl, ethyl, n-propyl, isopropyl, n-butyl, isobutyl, sec-butyl, tent-butyl, or allyl.

“Alkanyl” by itself or as part of another substituent refers to a saturated branched or straight-chain alkyl radical derived by the removal of one hydrogen atom from a single carbon atom of a parent alkane. Examples of alkanyl groups include, but are not limited to, methanyl, ethanyl, propanyls such as propan-1-yl, propan-2-yl (isopropyl), etc.; butanyls such as butan-1-yl, butan-2-yl (sec-butyl), 2-methyl-propan-1-yl (isobutyl), 2-methyl-propan-2-yl (t-butyl), etc.; and the like.

“Alkenyl” by itself or as part of another substituent refers to an unsaturated branched or straight-chain alkyl radical having at least one carbon-carbon double bond derived by the removal of one hydrogen atom from a single carbon atom of a parent alkene. The group may be in either the cis or trans conformation about the double bond(s). Examples of alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like.

“Alkynyl” by itself or as part of another substituent refers to an unsaturated branched or straight-chain alkyl radical having at least one carbon-carbon triple bond derived by the removal of one hydrogen atom from a single carbon atom of a parent alkyne. Examples of alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyloxy” by itself or as part of another substituent refers to a radical —OC(O)R³³, where R³³ is alkyl, cycloalkyl, cycloheteroalkyl, aryl, arylalkyl, heteroalkyl, heteroaryl, or heteroarylalkyl, which may be substituted, as defined herein. Examples of acyloxy groups include, but are not limited to, acetoxy, isobutyroyloxy, benzoyloxy, and phenylacetoxy.

“Alkoxy” by itself or as part of another substituent refers to a radical —OR³⁴ where R³⁴ is alkyl, cycloalkyl, cycloalkylalkyl, aryl, or arylalkyl, which may be substituted, as defined herein. Examples of alkoxy groups include, but are not limited to, methoxy, ethoxy, propoxy, butoxy, and cyclohexyloxy.

“Alkoxycarbonyl” by itself or as part of another substituent refers to a radical —C(O)OR³⁵ where R³⁵ is an alkyl or substituted alkyl group, as defined herein. Examples of alkoxycarbonyl groups include, but are not limited to, methoxycarbonyl, ethoxycarbonyl, propoxycarbonyl, and butoxycarbonyl. In certain embodiments, an alkoxycarbonyl group is C₁₋₃ alkoxycarbonyl.

“Alkoxycarbonylamino” by itself or as part of another substituent refers to a radical —NR³⁶C(O)—OR³⁷ where R³⁶ represents an alkyl, substituted alkyl, cycloalkyl, or substituted cycloalkyl group and R³⁷ is alkyl, cycloalkyl, cycloheteroalkyl, aryl, arylalkyl, heteroalkyl, heteroaryl, or heteroarylalkyl, which may be substituted, as defined herein. Examples of alkoxycarbonylamino groups include, but are not limited to, methoxycarbonylamino, tert-butoxycarbonylamino, and benzyloxycarbonylamino.

“Alkoxycarbonyloxy” by itself or as part of another substituent refers to a radical —OC(O)—OR³⁸ where R³⁸ is an alkyl, substituted alkyl, cycloalkyl, or substituted cycloalkyl group, as defined herein. Examples of alkoxycarbonyloxy groups include, but are not limited to, methoxycarbonyloxy, ethoxycarbonyloxy, and cyclohexyloxycarbonyloxy.

“Alkylamino” by itself or as part of another substituent refers to a radical —NHR³⁹ where R³⁹ is an alkyl, substituted alkyl, cycloalkyl, or substituted cycloalkyl group, as defined herein. In certain embodiments, an alkylamino group is C₁₋₃ alkylamino.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. In certain embodiments, an aryl group may have from 5 to 20 carbon atoms, and in certain embodiments, from 5 to 12 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein. In certain embodiments, aryl is C₆₋₁₀ aryl or phenyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, and 2-naphthophenylethan-1-yl. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂. In certain embodiments, arylalkyl is C₇₋₁₆ arylalkyl or benzyl.

“Aryldialkylsilyl” by itself or as part of another substituent refers to the radical —SiR⁴⁰R⁴¹R⁴² where one of R⁴⁰, R⁴¹, and R⁴² is aryl or substituted aryl as defined herein and the other two of R⁴⁰, R⁴¹, and R⁴² are alkyl or substituted alkyl, as defined herein. In certain embodiments, an aryldialkylsilyl group is C₇₋₁₄ aryldialkylsilyl.

“Bioavailability” refers to the amount of a drug that reaches the systemic circulation of a patient following administration of the drug or prodrug thereof to the patient and may be determined by evaluating, for example, the plasma or blood concentration-versus-time profile for a drug. Parameters useful in characterizing a plasma or blood concentration-versus-time curve include the area under the curve (AUC), the time to maximum concentration (Tₘₐₓ), and the maximum drug concentration (Cₘₐₓ), where Cₘₐₓ is the maximum concentration of a drug in the plasma or blood of a patient following administration of a dose of the drug or prodrug thereof to the patient, and Tₘₐₓ is the time to the maximum concentration (Cₘₐₓ) of a drug in the plasma or blood of a patient following administration of a dose of the drug or prodrug thereof to the patient.

“Carbamoyl” by itself or as part of another substituent refers to the radical —C(O)NR⁴³R⁴⁴ where R⁴³ and R⁴⁴ are independently hydrogen, alkyl, substituted alkyl, cycloalkyl, substituted cycloalkyl, aryl, or substituted aryl, as defined herein.

“Carbamoyloxy” by itself or as part of another substituent refers to a radical OC(O)NR⁴⁵R⁴⁶ where R⁴⁵ and R⁴⁶ are independently selected from hydrogen, alkyl, cycloalkyl, cycloheteroalkyl, aryl, arylalkyl, heteroalkyl, heteroaryl, and heteroarylalkyl, which may be substituted, as defined herein, or R⁴⁵ and R⁴⁶ together with the atoms to which they are bonded form a cycloheteroalkyl or heteroaryl ring.

“Cleave” refers to breakage of chemical bonds and is not limited to chemical or enzymatic reactions or mechanisms unless clearly intended by the context.

“Compounds” of Formula (I) disclosed herein include any specific compounds within these formulae. Compounds may be identified either by their chemical structure and/or chemical name. When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan. Compounds of Formula (I) include, but are not limited to, optical isomers of compounds of Formula (I), racemates thereof, and other mixtures thereof. In such embodiments, a single enantiomer or diastereomer, i.e., optically active form can be obtained by asymmetric synthesis or by resolution of the racemates. Resolution of the racemates may be accomplished, for example, by conventional methods such as crystallization in the presence of a resolving agent, or chromatography using, for example, chiral stationary phases. In addition, compounds of Formula (I) include Z- and E-forms (or cis- and trans-forms) of compounds with double bonds.

Compounds of Formula (I) may also exist in several tautomeric forms including the enol form, the keto form, and mixtures thereof. Accordingly, the chemical structures depicted herein encompass all possible tautomeric forms of the illustrated compounds. Compounds of Formula (I) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds as referred to herein may be free acid, hydrated, solvated, or N-oxides. Certain compounds may exist in multiple crystalline, co-crystalline, or amorphous forms. Compounds of Formula (I) include pharmaceutically acceptable salts thereof, or pharmaceutically acceptable solvates of the free acid form of any of the foregoing, as well as crystalline forms of any of the foregoing.

Compounds of Formula (I) also include solvates. A solvate refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Cycloalkoxycarbonyl” by itself or as part of another substituent refers to a radical —C(O)OR⁴⁷ where R⁴⁷ represents an cycloalkyl or substituted cycloalkyl group as defined herein. Examples of cycloalkoxycarbonyl groups include, but are not limited to, cyclobutyloxycarbonyl and cyclohexyloxycarbonyl.

“Cycloalkyl” by itself or as part of another substituent refers to a partially saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, and cyclohexane. In certain embodiments, a cycloalkyl group is C₃₋₁₅ cycloalkyl, and in certain embodiments. C₅₋₁₂ cycloalkyl. In certain embodiments, a cycloalkyl group is C₃₋₇ cycloalkyl or cyclohexyl.

“Cycloheteroalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S and Si. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, and quinuclidine.

“Dialkylamino” by itself or as part of another substituent refers to the radical —NR⁴⁸R⁴⁹ where R⁴⁸ and R⁴⁹ are independently alkyl, substituted alkyl, cycloalkyl, substituted cycloalkyl, cycloheteroalkyl, substituted cycloheteroalkyl, arylalkyl, substituted arylalkyl, heteroalkyl, substituted heteroalkyl, heteroarylalkyl, or substituted heteroarylalkyl, or R⁴⁸ and R⁴⁹ together with the nitrogen to which they are attached form a cycloheteroalkyl or substituted cycloheteroalkyl ring. In certain embodiments, a dialkylamino group is C₁₋₃ dialkylamino.

“Heteroalkyl” by itself or as part of another substituent refer to an alkyl group in which one or more of the carbon atoms (and any associated hydrogen atoms) are independently replaced with the same or different heteroatomic groups. Examples of heteroatomic groups include, but are not limited to, —O—, —S—, —O—O—, —S—S—, —O—S—, —NR⁵⁰R⁵¹—, ═N—N═, —N═N—, —N═N—NR⁵²R⁵³, —PR⁵⁴—, —P(O)₂—, —POR⁵⁵—, —O—P(O)₂—, —SO—, —SO₂—, —SnR⁵⁶R⁵⁷—, where R⁵⁰, R⁵¹, R⁵², R⁵³, R⁵⁴, R⁵⁵, R⁵⁶, and R⁵⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkenyl,” “heteroalkenyl,” or “heteroalkynyl” is used.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which may be aromatic or non-aromatic in which at least one ring atom is a heteroatom. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and xanthene. In certain embodiments, a heteroaryl group is from 5- to 20-membered heteroaryl, and in certain embodiments from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Immediately preceding embodiments” means the embodiments disclosed in the same paragraph.

“Parent aromatic ring system” refers to an unsaturated cyclic or polycyclic ring system having a conjugated π electron system. Included within the definition of “parent aromatic ring system” are fused ring systems in which one or more of the rings are aromatic and one or more of the rings are saturated or unsaturated, such as, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, pyrimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and xanthene.

“Pharmaceutical composition” refers to at least one compound and a pharmaceutically acceptable vehicle with which the compound is administered to a patient.

“Pharmaceutically acceptable” refers to approved or approvable by a regulatory agency of the Federal or a state government or listed in the U.S. Pharmacopoeia or other generally recognized pharmacopoeia for use in animals, including humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

The term “pharmaceutically acceptable salt” includes hydrates and other solvates, as well as salts in crystalline or non-crystalline form.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound of Formula (I) can be administered to a patient and which does not destroy the pharmacological activity thereof and which is nontoxic when administered in doses sufficient to provide a therapeutically effective amount of the compound.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation to which the prodrug is administered or the acidic conditions of the stomach, or the agent may be supplied exogenously. For example, the promoiety of 4-({[1-(2-methylpropanoyloxy)ethoxy]carbonylamino}methyl)-cyclohexanecarboxylic acid is:

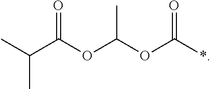

“Subject” includes mammals, such as for example, humans. The terms “subject” and“patient” are used interchangeably.

“Substantially one enantiomer” refers to a compound containing 1 or more stereogenic centers such that the enantiomeric excess (e.e.) of the compound is at least about 90%, in certain embodiments greater than about 95%, in certain embodiments greater than about 98%, and in certain embodiments greater than about 99%.

“Substituted” refers to a group in which one or more hydrogen atoms are independently replaced with the same or different substituent(s). Example of substituents include, but are not limited to, -M, —R⁶⁰, —O⁻(—OH), ═O, —OR⁶⁰, —SR⁶⁰, —S⁻(—SH), ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is a halogen; R⁶⁰, R⁶¹, R⁶², and R⁶³ are independently selected from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, and substituted heteroaryl, or when bonded to a nitrogen atom, R⁶⁰ and R⁶¹ together with the nitrogen atom to which they are bonded Corm a cycloheteroalkyl or substituted cycloheteroalkyl ring; and R⁶² and R⁶³ are independently selected from hydrogen, alkyl, substituted alkyl, aryl, cycloalkyl, substituted cycloalkyl, cycloheteroalkyl, substituted cycloheteroalkyl, aryl, substituted aryl, heteroaryl, and substituted heteroaryl, or when bonded to a nitrogen atom, R⁶² and R⁶³ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S—, —NR⁶⁰R⁶¹, —NR⁶⁰, —CF₃, —CN, —OCN, —SCN, —NO, —NO₂, ═N₂, —N₃, —S(O)₂R⁶⁰, —OS(O₂)O⁻, —OS(O)₂R⁶⁰)₂, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, and —NR⁶²C(O)NR⁶⁰R⁶¹, where R⁶⁰, R⁶¹, and R⁶² are as defined above. In other embodiments, substituents may be chosen from -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁼, where R⁶⁰ and R⁶¹ are as defined above. In yet other embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, and —C(O)O⁻, where R⁶⁰ and R⁶¹ are as defined above. In certain embodiments, each substituent is independently selected from C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino, as defined herein.

“Sustained release” refers to release of a therapeutic or preventive amount of a drug or an active metabolite thereof over a period of time that is longer than that of an immediate release formulation of the drug. For oral formulations, the term “sustained release” typically means release of the drug within the gastrointestinal tract lumen over a time period ranging, for example, from about 2 to about 30 hours, and in certain embodiments, over a time period ranging from about 4 to about 24 hours. Sustained release formulations achieve therapeutically effective concentrations of the drug in the systemic circulation over a prolonged period of time relative to that achieved by oral administration of an immediate release formulation of the drug.

“Thioalkyl” by itself or as part of another substituent refers to a radical —SR⁶⁷ where R⁶⁷ is alkyl or substituted alkyl, as defined herein. In certain embodiments, a thioalkyl group is C₁₋₃ thioalkyl.

“Treating” or “treatment” of bleeding refers to reducing, minimizing, and/or preventing bleeding in a patient. In certain embodiments, “treating” or “treatment” refers to reducing, minimizing, and/or preventing bleeding in a patient who is not experiencing bleeding, a patient who is bleeding, and/or to a patient who was bleeding. In certain embodiments, “treating” or “treatment” refers to administering a compound provided by the present disclosure prophylactically in anticipation of potential bleeding.

“Therapeutically effective amount” refers to the amount of a compound that, when administered to a patient for treating bleeding in a patient, is sufficient to reduce, minimize, and/or prevent bleeding. A “therapeutically effective amount” can vary depending, for example, on the compound, the nature or cause of the bleeding, severity of the bleeding, the age, weight, and/or health of the patient to be treated, and the judgment of the prescribing physician. An appropriate amount in any given instance can be ascertained by those skilled in the art or capable of determination by routine experimentation. The terms “therapeutically effective amount” and “prophylactically effective amount” are used interchangeably.

“Trialkylsilyl” by itself or as part of another substituent refers to a radical —SiR⁶⁸R⁶⁹R⁷⁰ where R⁶⁸, R⁶⁹, and R⁷⁰ are independently selected from alkyl and substituted alkyl, as defined herein. In certain embodiments, a trialkylsilyl group is C₃₋₁₂ trialkylsilyl.

“Wound” refers to any break in the skin or an organ of a subject. A “wound” may result from any cause, intentional or unintentional, including trauma, accident, surgery, disease, and/or chemicals including drugs. “Bleeding caused by a wound” refers to bleeding the occurs as the direct result of the injury such as from tissue or organs of the wound, as well as bleeding the occurs in peripheral or other tissue of the subject caused by an imbalance in homeostasis resulting from, exacerbated by, and/or initiated by the wound.

Reference is now made in detail to certain embodiments of compounds, compositions, and methods according to the present disclosure. The disclosed embodiments are not intended to be limiting of the claims. To the contrary, the claims are intended to cover all alternatives, modifications, and equivalents.

In certain embodiments of the present disclosure, acyloxyalkyl carbamate prodrugs of tranexamic acid are selected from a compound of Formula (I):

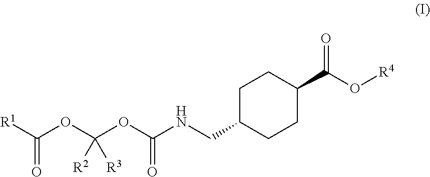

or a pharmaceutically acceptable salt thereof, wherein:

R¹ is selected from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently selected from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and

R⁴ is selected from hydrogen, alkyl, substituted alkyl, aryl, substituted aryl, arylalkyl, substituted arylalkyl, aryldialkylsilyl, substituted aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, trialkylsilyl, and substituted trialkylsilyl.

In certain embodiments of a compound of Formula (I), the compound, which when administered in the intestinal lumen of a patient is absorbed to a sufficient extent so as to achieve a bioavailability of trans-4-(aminomethyl)-cyclohexanecarboxylic acid at least 2-fold greater than the bioavailability of trans-4-(aminomethyl)-cyclohexanecarboxylic acid achieved when trans-4-(aminomethyl)-cyclohexanecarboxylic acid itself is administered in the intestinal lumen of the patient.

In certain embodiments of a compound of Formula (I), R¹ is selected from C₁₋₆ alkyl, substituted C₁₋₆ alkyl, C₆₋₁₀ aryl, substituted C₆₋₁₀ aryl, C₃₋₇ cycloalkyl, substituted C₃₋₇ cycloalkyl, C₇₋₁₆ arylalkyl, and C₇₋₁₆ substituted arylalkyl. In certain of the immediately preceding embodiments, the substituent group of R¹ is selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), R⁴ is selected from C₁₋₄ alkyl, substituted C₁₋₄ alkyl, phenyl, substituted phenyl, cyclohexyl, and substituted cyclohexyl. In certain of the immediately preceding embodiments, each substituent group of R¹ is independently selected from at least one of C₁₋₃ alkyl, —OH, NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), each substituent group of R¹ is independently selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), R¹ is selected from methyl, ethyl, n-propyl, isopropyl, n-butyl, isobutyl, sec-butyl, tert-butyl, phenyl, o-tolyl, and cyclohexyl.

In certain embodiments of a compound of Formula (I), R⁴ is selected from hydrogen, C₁₋₆ alkyl, substituted C₁₋₆ alkyl, C₃₋₇ cycloalkyl, substituted C₃₋₇ cycloalkyl, C₆₋₁₀ aryl, substituted C₆₋₁₀ aryl, C₇₋₁₆ arylalkyl, substituted C₇₋₁₆ arylalkyl, C₃₋₁₇ trialkylsilyl, and C₇₋₁₄ aryldialkylsilyl. In certain of the immediately preceding embodiments, each substituent group of R⁴ is independently selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), R⁴ is selected from hydrogen, methyl, ethyl, tort-butyl, allyl, benzyl, 4-methoxybenzyl, diphenylmethyl, triphenylmethyl, trimethylsilyl, triethylsilyl, triisopropylsilyl, tert-butyldimethylsilyl, and phenyldimethylsilyl.

In certain embodiments of a compound of Formula (I), R⁴ is selected from hydrogen, allyl, benzyl, and trimethylsilyl.

In certain embodiments of a compound of Formula (I), R⁴ is hydrogen.

In certain embodiments of a compound of Formula (I), R² and R³ are independently selected from hydrogen, C₁₋₆ alkyl, substituted C₁₋₆ alkyl, C₆₋₁₀ aryl, substituted C₆₋₁₀ aryl, C₃₋₇ cycloalkyl, and substituted C₃₋₇ cycloalkyl. In certain of the immediately preceding embodiments, each substituent group of R² and/or R³ is independently selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), R² and R³ are independently selected from hydrogen, methyl, ethyl, n-propyl, isopropyl, phenyl, and cyclohexyl.

In certain embodiments of a compound of Formula (I), R² is hydrogen, and R³ is selected from hydrogen, methyl, ethyl, n-propyl, isopropyl, phenyl, and cyclohexyl. In certain embodiments of a compound of Formula (I), R² is hydrogen, and R³ is selected from methyl, ethyl, n-propyl, isopropyl, phenyl, and cyclohexyl.

In certain embodiments of a compound of Formula (I), R⁴ is selected from C₁₋₆ alkyl, substituted C₁₋₆ alkyl, C₆₋₁₀ aryl, substituted C₆₋₁₀ aryl, C₃₋₇ cycloalkyl, substituted C₃₋₇ cycloalkyl, C₇₋₁₆ arylalkyl, and C₇₋₁₆ substituted arylalkyl, and R² and R³ are independently selected from hydrogen, C₁₋₆ alkyl, substituted C₁₋₆ alkyl, C₆₋₁₀ aryl, substituted C₆₋₁₀ aryl, C₃₋₇ cycloalkyl, and substituted C₃₋₇ cycloalkyl. In certain of the immediately preceding embodiments, each substituent group of R¹ is independently selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I). R¹ is selected from methyl, ethyl, n-propyl, isopropyl, n-butyl, isobutyl, sec-butyl, tert-butyl, phenyl, o-tolyl, and cyclohexyl, R² is hydrogen, and R³ is selected from hydrogen, methyl, ethyl, n-propyl, isopropyl, phenyl, and cyclohexyl. In certain embodiments of a compound of Formula (I), R¹ is selected from methyl, ethyl, n-propyl, isopropyl, n-butyl, isobutyl, sec-butyl, tert-butyl, phenyl, o-tolyl, and cyclohexyl, R² is hydrogen, and R³ is selected from methyl, ethyl, n-propyl, isopropyl, phenyl, and cyclohexyl.

In certain embodiments of a compound of Formula (I), R⁴ is hydrogen.

In certain embodiments of a compound of Formula (I), each of R² and R³ is other than hydrogen. When each of R² and R³ is hydrogen, a metabolite of certain acyloxyalkylcarbamate promoieties may be formaldehyde. In some embodiments for methods of treatment comprising administering large amounts of a compound of Formula (I) it may be desirable that the amount of toxic metabolites of the promoiety such as formaldehyde be minimized or eliminated.

In certain embodiments of a compound of Formula (I), R¹ is selected from C₁₋₆ alkyl, substituted C₁₋₆ alkyl, C₆₋₁₀ aryl, substituted C₆₋₁₀ aryl, C₃₋₇ cycloalkyl, substituted C₃₋₇ cycloalkyl, C₇₋₁₆ arylalkyl, and C₇₋₁₆ substituted arylalkyl;

R² and R³ are independently selected from hydrogen, C₁₋₆ alkyl, substituted C₁₋₆ alkyl, C₆₋₁₀ aryl, substituted C₆₋₁₀ aryl, C₃₋₇ cycloalkyl, and substituted C₃₋₇ cycloalkyl;

R⁴ is selected from hydrogen, C₁₋₆ alkyl, substituted C₁₋₆ alkyl, C₃₋₇ cycloalkyl, substituted C₃₋₇ cycloalkyl, C₆₋₁₀ aryl, substituted C₆₋₁₀ aryl, C₇₋₁₆ arylalkyl, substituted C₇₋₁₆ arylalkyl, C₃₋₁₂ trialkylsilyl, and C₇₋₁₄ aryldialkylsilyl; and

wherein each substituent group is independently selected from at least one of C₁₋₃ alkyl, —OH, —NH₂, —SH, C₁₋₃ alkoxy, C₁₋₃ acyl, C₁₋₃ thioalkyl, C₁₋₃ alkoxycarbonyl, C₁₋₃ alkylamino, and C₁₋₃ dialkylamino.

In certain embodiments of a compound of Formula (I), R¹ is selected from methyl, ethyl, n-propyl, isopropyl, n-butyl, isobutyl, sec-butyl, tert-butyl, phenyl, o-tolyl, and cyclohexyl; R² is hydrogen; R³ is selected from methyl, ethyl, n-propyl, isopropyl, phenyl, and cyclohexyl; and R⁴ is hydrogen.

In certain embodiments of a compound of Formula (I), R¹ is selected from C₁₋₄ alkyl, phenyl, o-tolyl, and cyclohexyl; R² is hydrogen; R³ is selected from C₁₋₃ alkyl, phenyl, and cyclohexyl; and R⁴ is selected from hydrogen, C₁₋₄ alkyl, benzyl, 4-methoxybenzyl, diphenylmethyl, triphenylmethyl, trimethylsilyl, triethylsilyl, triisopropylsilyl, tert-butyldimethylsilyl, and phenyldimethylsilyl.

In certain embodiments of a compound of Formula (I), R¹ is selected from isopropyl, isobutyl, and phenyl; R² is hydrogen; R³ is selected from methyl and isopropyl; and R⁴ is hydrogen.

In certain embodiments of a compound of Formula (I), R¹ is isopropyl; one of R² and R³ is hydrogen and the other of R² and R³ is methyl; and R⁴ is hydrogen.

In certain embodiments of a compound of Formula (I), the compound is selected from (+)-trans-4-({[(1S)-1-(2-methylpropanoyloxy)ethoxy]carbonylamino}methyl)-cyclohexanecarboxylic acid; (−)-trans-4-({[(1R)-1-(2-methylpropanoyloxy)ethoxy]carbonylamino}methyl)-cyclohexanecarboxylic acid; a pharmaceutically acceptable salt of any of the foregoing; and mixtures of any of the foregoing.

In certain embodiments of a compound of Formula (I), the compound is trans-4-{[1-(2-methylpropanoyloxy)ethoxycarbonyl]aminomethyl}-cyclohexanecarboxylic acid or a pharmaceutically acceptable salt thereof.

In certain embodiments of a compound of Formula (I), the compound is selected from sodium trans-4-({[(1S)-1-(2-methylpropanoyloxy)ethoxy]carbonylamino}methyl)-cyclohexanecarboxylate; sodium trans-4-({[(1R)-1-(2-methylpropanoyloxy)ethoxy]carbonylamino}methyl)-cyclohexanecarboxylate; and mixtures thereof.

In certain embodiments of a compound of Formula (I), the compound is sodium trans-4-({[1-(2-methylpropanoyloxy)ethoxy]carbonylamino}methyl)-cyclohexanecarboxylate.

In certain embodiments of a compound of Formula (I), the compound is selected from trans-4-{[1-(2-methylpropanoyloxy)-2-methylpropoxycarbonyl]aminomethyl}-cyclohexanecarboxylic acid and a pharmaceutically acceptable salt thereof.

In certain embodiments of a compound of Formula (I), the compound is selected from trans-4-{[1-(benzoyloxy)ethoxycarbonyl]aminomethyl}-cyclohexanecarboxylic acid and a pharmaceutically acceptable salt thereof.

In certain embodiments of a compound of Formula (I), the compound is selected from:
trans-4-{[(2-methylpropanoyloxy)methoxycarbonyl]aminomethyl}-cyclohexanecarboxylic acid;
trans-4-{[(2,2-dimethylpropanoyloxy)methoxycarbonyl]-aminomethyl}-cyclohexanecarboxylic acid;
trans-4-{[(3-methylbutanoyloxy)methoxycarbonyl]aminomethyl}-cyclohexanecarboxylic acid;
trans-4-{[(benzoyloxy)methoxycarbonyl]aminomethyl}-cyclohexanecarboxylic acid;
trans-4-{[1-(2-methylpropanoyloxy)ethoxycarbonyl]aminomethyl}-cyclohexanecarboxylic acid;
trans-4-{[1-(2-methylpropanoyloxy)propoxycarbonyl]aminomethyl}-cyclohexanecarboxylic acid;
trans-4-{[1-(2-methylpropanoyloxy)-2-methylpropoxycarbonyl]aminomethyl}-cyclohexanecarboxylic acid;
trans-4-{[1-(benzoyloxy)-2-methylpropoxycarbonyl]aminomethyl}-cyclohexanecarboxylic acid;
trans-4-{[1-(cyclohexylcarbonyloxy)-2-methylpropoxycarbonyl]aminomethyl}cyclohexanecarboxylic acid;
trans-4-{[1-(pentanoyloxy)-2-methylpropoxycarbonyl]aminomethyl}-cyclohexanecarboxylic acid;
trans-4-{[1-(propanoyloxy)-2-methylpropoxycarbonyl]aminomethyl}-cyclohexanecarboxylic acid;
trans-4-{[1-(butanoyloxy)ethoxycarbonyl]aminomethyl}-cyclohexanecarboxylic acid;
trans-4-{[1-(pentanoyloxy)ethoxycarbonyl]aminomethyl}-cyclohexanecarboxylic acid;
trans-4-{[1-(3-methylbutanoyloxy)ethoxycarbonyl]aminomethyl}-cyclohexanecarboxylic acid;
trans-4-{[1-(2,2-dimethylpropanoyloxy)ethoxycarbonyl]aminomethyl}-cyclohexanecarboxylic acid;
trans-4-{[1-(cyclohexylcarbonyloxy)ethoxycarbonyl]aminomethyl}-cyclohexanecarboxylic acid;
trans-4-{[1-(benzoyloxy)ethoxycarbonyl]aminomethyl}-cyclohexanecarboxylic acid;
trans-4-{[1-(2-methylbenzoyloxy)ethoxycarbonyl]aminomethyl}-cyclohexanecarboxylic acid;
trans-4-{[1-(butanoyloxy)butoxycarbonyl]aminomethyl}-cyclohexanecarboxylic acid;
trans-4-{[1-(2-methylpropanoyloxy)butoxycarbonyl]aminomethyl}-cyclohexanecarboxylic acid;
trans-4-{[1-(3-methylbutanoyloxy)butoxycarbonyl]aminomethyl}cyclohexanecarboxylic acid;
trans-4-{[1-(2,2-dimethylpropanoyloxy)butoxycarbonyl]aminomethyl}-cyclohexanecarboxylic acid;
trans-4-{[1-(benzoyloxy)butoxycarbonyl]aminomethyl}-cyclohexanecarboxylic acid;
trans-4-{[1-(propanoyloxy)propoxycarbonyl]aminomethyl}-cyclohexanecarboxylic acid;
trans-4-{[1-(butanoyloxy)propoxycarbonyl]aminomethyl}-cyclohexanecarboxylic acid;
trans-4-{[1-(2,2-dimethylpropanoyloxy)propoxycarbonyl]aminomethyl}-cyclohexanecarboxylic acid;
trans-4-{[1-(benzoyloxy)propoxycarbonyl]aminomethyl}-cyclohexanecarboxylic acid;
trans-4-{[1-(butanoyloxy)-2-methylpropoxycarbonyl]aminomethyl}-cyclohexanecarboxylic acid;
trans-4-{[1-(butanoyloxy)-1-cyclohexylmethoxycarbonyl]aminomethyl}-cyclohexanecarboxylic acid;
trans-4-{[1-(2-methylpropanoyloxy)-1-cyclohexylmethoxycarbonyl]aminomethyl}-cyclohexanecarboxylic acid;
trans-4-{[1-(acetoxy)butoxycarbonyl]aminomethyl}-cyclohexanecarboxylic acid;
trans-4-{[1-(propanoyloxy)butoxycarbonyl]aminomethyl}-cyclohexanecarboxylic acid;
trans-4-{[1-(acetoxy)-2-methylpropoxycarbonyl]aminomethyl}-cyclohexanecarboxylic acid;
trans-4-{[1-(3-methylbutanoyloxy)-2-methylpropoxycarbonyl]aminomethyl}-cyclohexanecarboxylic acid;
trans-4-{[1-(2,2-dimethylpropanoyloxy)-2-methylpropoxycarbonyl]aminomethyl}-cyclohexanecarboxylic acid;
trans-4-{[1-(acetoxy)ethoxycarbonyl]aminomethyl}-cyclohexanecarboxylic acid;
trans-4-{[1-(propanoyloxy)ethoxycarbonyl]aminomethyl}-cyclohexanecarboxylic acid; and

a pharmaceutically acceptable salt of any of the foregoing.

Methods of synthesizing acyloxyalkyl carbamate prodrugs of tranexamic acid provided by the present disclosure are disclosed in Zerangue et al., U.S. Pat. No. 7,351,740, which is incorporated by reference herein.

Pharmaceutical compositions provided by the present disclosure may comprise a therapeutically effective amount of a compound of Formula (I) together with a suitable amount of one or more pharmaceutically acceptable vehicles so as to provide a composition for proper administration to a patient. Suitable pharmaceutical vehicles are described in the art.

In certain embodiments, a tranexamic prodrug of Formula (I) can be provided to a patient by topical administration. For example, a pharmaceutical composition comprising at least on compound of Formula (I) and at least one pharmaceutically acceptable topical vehicle can be formulated in the form of a cream, lotion, ointment, solution, aerosol, spray and the like. The topical formulation can be applied to a surface area of a patient to be treated, for example, by spreading or spraying. The surface area of a patient to be treated can be an area exhibiting bleeding from a wound. In certain embodiments, a tranexamic prodrug of Formula (I) can be provided to a patient by topical administration prophylactically.

In certain embodiments, a compound of Formula (I) may be incorporated into pharmaceutical compositions to be administered orally. Oral administration of such pharmaceutical compositions may result in uptake of a compound of Formula (I) throughout the intestine and entry into the systemic circulation. Such oral compositions may be prepared in a manner known in the pharmaceutical art and comprise a compound of Formula (I) and at least one pharmaceutically acceptable vehicle. Oral pharmaceutical compositions may include a therapeutically effective amount of a compound of Formula (I) and a suitable amount of a pharmaceutically acceptable vehicle, so as to provide an appropriate form for administration to a patient.

Pharmaceutical compositions comprising a compound of Formula (I) may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds of Formula (I) or crystalline forms thereof and one or more pharmaceutically acceptable vehicles into formulations that can be used pharmaceutically. Proper formulation is dependent upon the route of administration chosen. Pharmaceutical compositions provided by the present disclosure may take the form of solutions, suspensions, emulsion, tablets, pills, pellets, capsules, capsules containing liquids, powders, sustained-release formulations, suppositories, emulsions, aerosols, sprays, suspensions, or any other form suitable for administration to a patient.

Pharmaceutical compositions provided by the present disclosure may be formulated in a unit dosage form. A unit dosage form refers to a physically discrete unit suitable as a unitary dose for patients undergoing treatment, with each unit containing a predetermined quantity of a compound of Formula (I) calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

Pharmaceutical compositions comprising a compound of Formula (I) may be formulated for immediate release.

In certain embodiments, an oral dosage form provided by the present disclosure may be a controlled release dosage form. Controlled delivery technologies can improve the absorption of a drug in a particular region or regions of the gastrointestinal tract. Controlled drug delivery systems may be designed to deliver a drug in such a way that the drug level is maintained within a therapeutically effective window and effective and safe blood levels are maintained for a period as long as the system continues to deliver the drug at a particular rate. Controlled drug delivery may produce substantially constant blood levels of a drug over a period of time as compared to fluctuations observed with immediate release dosage forms. For some drugs, maintaining a constant blood and tissue concentration throughout the course of therapy is the most desirable mode of treatment. Immediate release of drugs may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

An appropriate oral dosage form for a particular pharmaceutical composition provided by the present disclosure may depend, at least in part, on the gastrointestinal absorption properties of a compound of Formula (I) the stability of a compound of Formula (I) in the gastrointestinal tract, the pharmacokinetics of a compound of Formula (I), and the intended therapeutic profile. An appropriate controlled release oral dosage form may be selected for a particular compound of Formula (I). For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Certain compounds are absorbed at least in part from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

In certain embodiments, pharmaceutical compositions provided by the present disclosure may be practiced with dosage forms adapted to provide sustained release of a compound of Formula (I) upon oral administration. Sustained release oral dosage forms may be used to release drugs over a prolonged time period and are useful when it is desired that a drug or drug form be delivered to the lower gastrointestinal tract. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

An appropriate dose of a compound of Formula (I) or pharmaceutical composition comprising a compound of Formula (I) may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

Compounds of Formula (I) may be administered to reduce or minimized bleeding in a patient who either anticipates bleeding such as during surgery or traumatic injury or who is bleeding. Compounds of Formula (I) may be effectively used prophylactically to reduce or minimize bleeding such as perioperative bleeding and in bleeding due to traumatic injury.

Bleeding refers to extravasation of blood from any component of the circulatory system and includes unwanted and uncontrolled bleeding in connection with surgery, trauma, or other forms of tissue damage, as well as unwanted bleedings in subjects having bleeding disorders. Bleeding may occur in subjects having a basically normal coagulation system but experiencing a (temporary) coagulopathy, as well as in subjects having congenital or acquired coagulation bleeding disorders. Compounds provided by the present disclosure may be used to control bleeding in patients having a bleeding disorder or may be used to control bleeding occurring in subjects with a normally functioning blood clotting cascade (no clotting factor deficiencies or inhibitors against any of the coagulation factors)

In certain embodiments, a therapeutically effective amount of a tranexamic acid prodrug, such as a compound of Formula (I), may be administered to a subject to treat bleeding caused by a wound prior to incurring the wound. In certain embodiments, a therapeutically effective amount of a tranexamic acid prodrug, such as a compound of Formula (I), may be administered to a subject to treat bleeding caused by a wound at least about 1 hour, at least about 2 hours, at least about 4 hours, at least about 8 hours, at least about 12 hours, or at least about 24 hours prior to incurring the wound. A compound may be administered prior to incurring an intentional wound such as, for example, prior to elective surgery, or prior to incurring an unintentional wound such, for example, prior to traumatic injury.

In certain embodiments, a compound of Formula (I), a pharmaceutically acceptable salt thereof, or a pharmaceutical compositions of any of the foregoing may be used to treat bleeding resulting from surgery. Examples of surgical procedures in which methods provided by the present disclosure can be useful include brain surgery, nasal surgery such as rhinoplasty, septoplasty, turbinectomy and functional endoscopy sinus surgery; orthognathic surgery; prostatectomy; splenectomy; gall bladder surgery; gynecological surgery such as oophorectomy, Cesarean section, and hysterectomy; liver transplant; eye surgery; dental surgery; laparoscopic surgery; cancer surgery including bladder cancer, lung cancer, and esophageal cancer; orthopedic surgery such as hip replacement, spinal fusion surgery, spinal surgery, scoliosis surgery, hip arthroplasty, and knee arthroplasty; and cardiac surgery such as coronary artery bypass surgery, and valve replacement surgery.

In certain embodiments, a compound of Formula (I), a pharmaceutically acceptable salt thereof, or a pharmaceutical composition of any of the foregoing may be administered perioperatively to a patient including before surgery, during surgery, and/or after surgery.

In certain embodiments, a compound of Formula (I), a pharmaceutically acceptable salt thereof, or a pharmaceutical composition of any of the foregoing may be administered prophylactically, before surgery, to treat bleeding during and/or following surgery (i.e., perioperative bleeding). In certain embodiments, prophylactic amounts of a compound of Formula (I) may be administered from about 1 to about 24 hours before surgery; from about 1 to about 12 hours before surgery; from about 1 to about 6 hours before surgery; and in certain embodiments, from about 1 to about 3 hours before surgery. In certain embodiments, prophylactic amounts of a compound of Formula (I) may be administered from about 1 day to about 3 days before surgery; from about 1 day to about 2 days before surgery; and in certain embodiments, about 1 day before surgery. In certain embodiments, a compound of Formula (I) may be administered at least about 2 hours before surgery, at least about 6 hours before surgery, at least about 12 hours before surgery, and in certain embodiments, at least about 24 hours before surgery.

Patients administered a compound of Formula (I) perioperatively may or may not have an underlying bleeding disorder.

Menorrhagia is defined as blood loss >80 mL per menstrual cycle and affects many women and represents a significant health problem. Prevalence rates are believed to be similar across the Western world, and in the U.K. at least one in 20 women aged between 34 and 49 years will consult their general practitioners because of menstrual disorders. Menorrhagia accounts for 60% of primary-care consultations for menstrual problems and 12% of all gynecology referrals (Peto et al., Fam. Pract., 1993, 10, 207-211; McPherson and Andersson, eds., Women's problems in general practice, Oxford: Oxford University Press, 1983, pp 21-41; Bradlow et al., Patterns of referral, Oxford: Oxford Health Services Research Unit, 1992). While various pathological mechanisms may contribute to the cause of menorrhagia, approximately 50% of women with heavy menstrual blood loss have no underlying anatomical or endocrinological abnormality. In such women fibrinolytic activity in utero is higher than in women with normal menstrual blood loss, with this increased fibrinolysis resulting from elevated levels of endometrium-derived plasmin and plasminogen activators (Gleeson, Am. J. Obstet. Gynecol., 1994, 171, 178-183; Dockeray et al., Eur. J. Obstet. Gynecol. Reprod. Biol., 1987, 309-318). Tranexamic acid is known to be useful in treating menorrhagia (Wellington and Wagstaff, Drugs 2003, 63(13), 1417-33).

In certain embodiments, a compound of Formula (I), a pharmaceutically acceptable salt thereof, or a pharmaceutical composition of any of the foregoing may be used to treat bleeding resulting from trauma. Traumatic injury includes, for example, abrasions, contusions, lacerations, incisions, gunshot wounds, blunt impact, and injury resulting from combat, including military combat and combat associated with law enforcement such as by police. For example, a compound of Formula (I) can be used by military combat personnel in order to reduce the risk of blood loss for those combatants who sustain field trauma, e.g., trauma from a projectile impact, an explosive blast, a vehicle accident, etc. For example, in certain combat situations, the compounds of Formula (I) can be used (i) for the prevention of bleeding/exsanguination from non-compressible trauma; (ii) in early/field treatment to reduce bleeding and the need for blood transfusions; and/or (iii) to complement standard medical help for more effective direct pressure, compression bandages, etc. from augmented clot stabilization in the field.

In certain embodiments, a compound of Formula (I), a pharmaceutically acceptable salt thereof, or a pharmaceutical composition of any of the foregoing may be administered prophylactically to treat bleeding in anticipation of possible trauma, such as prior to combat. For example, in military situations in which combat is anticipated and the potential for traumatic injury is significant, a compound of Formula (I) may be administered to the combatant at least about 2 hours, at least about 6 hours, at least about 12 hours, or at least about 24 hours prior to entering a combat situation. A compound of Formula (I) may continue to be administered as long as a significant potential for traumatic injury exists and as appropriate to provide a prophylactically effective plasma or blood concentration of tranexamic acid.

In certain embodiments, a compound of Formula (I), a pharmaceutically acceptable salt thereof, or a pharmaceutical compositions of any of the foregoing may be used to treat bleeding associated with bleeding disorders (Greaves and Watson, J Thrombosis Hemostasis 2007, 5(Suppl. 1), 167-174). A bleeding disorder can be any physiological defect of cellular or molecular origin that results in abnormal or pathological bleeding. A bleeding disorder may be congenital, acquired, or induced. Acquired bleeding disorders of primary hemostasis include bleeding due to pharmacological platelet inhibitors; clotting factor deficiencies such as hemophilia A, hemophilia B, hemophilia C, or deficiency of coagulation factors VII, IX, or XI; defective platelet function such as Glanzmann thombasthenia and Bernard-Doulier syndrome; thrombocytopenias; primary bone marrow diseases such as myeloproliferative, myelodysplastic, leukemic, and plasma cell dyscrasias; and severe renal failure. Acquired bleeding disorders of coagulation/fibrinolysis include hepatocellular failure, vitamin K deficiency, bleeding due to pharmacological anticoagulants, and coagulation factor inhibitors. Scurvy is another example of a mild bleeding disorder. Bleeding disorders include coagulopathy such as caused by a dilution of coagulation proteins, increased fibrinolysis and lowered number of platelets due to bleedings and/or tranfusions, such as in patients having multiple transfusions. Bleeding disorders further include inherited macrothrombocytopenias (platelet disorders) such as Bernard-Soulier syndrome, MYH9 gene-related disorders, macrothrobocytopenia and 22q11.2 deletion syndrome, gray platelet syndrome, Montreal platelet syndrome, benign Mediterranean macrothrobocytopenia, macrothrobocytopenia associated with mitral valve insufficiency, macrothrombocytopenia with platelet expression of glycophorin A, and macrothrombocytopenia with neutropenia.

In certain embodiments, a compound of Formula (I), a pharmaceutically acceptable salt thereof, or a pharmaceutical composition of any of the foregoing may be administered to treat hereditary thrombocytopenia syndromes including congenital amegakaryocytic thromobcytopeina (CAMT), thrombocytopenia absent radius syndrome. Fanconi anemia, Bernard-Soulier syndrome, May Hegglin anomaly, Grey platelet syndrome, or Alport syndrome.

In certain embodiments, a compound of Formula (I), a pharmaceutically acceptable salt thereof, or a pharmaceutical composition of any of the foregoing may be administered to treat thrombocytopenia induced by valproic acid, methotrexate, carboplatin, interferon, isotetinoin, 112 blockers, chemotherapeutic agents, or proton pump inhibitors.

In certain embodiments, a compound of Formula (I), a pharmaceutically acceptable salt thereof, or a pharmaceutical composition of any of the foregoing may be administered to treat thrombocytopenia characterized by increased platelet destruction such as idiopathic thrombocytopenic purpura, throbotic thrombocytopenic purpura, hemnolytic-uremic syndrome, disseminated intravascular coagulation, paroxysmal nocturnal hemoglohinuria, antiphospholipid syndrome, systemic lupus erythematosus, post transfusion purpura, neonatal alloimmune thrombocytopenia, hypersplenism, Dengue fever, or HIV-associated thrombocytopenia.

In certain embodiments, a compound of Formula (I), a pharmaceutically acceptable salt thereof, or a pharmaceutical composition of any of the foregoing may be administered to treat thrombocytopenia characterized by decreased platelet production including vitamin B12 or folic acid deficiency, leukemia or myelodysplastic syndrome, liver failure, sepsis and systemic viral or bacterial infection, and Dengue fever.

In certain embodiments, a compound of Formula (I), a pharmaceutically acceptable salt thereof, or a pharmaceutical composition of any of the foregoing may be administered to treat hyperfibrinolysis associated with leukemia, aplastic anemia, and surgery.

In certain embodiments, a compound of Formula (I), a pharmaceutically acceptable salt thereof, or a pharmaceutical composition of any of the foregoing may be administered to treat menorrhagia.

In certain embodiments, a compound of Formula (I), a pharmaceutically acceptable salt thereof, or a pharmaceutical composition of any of the foregoing may be administered to treat periodontal disease including gingivitis, periodontitis, and chronic progressive marginal periodontitis.

In certain embodiments, a compound of Formula (I), a pharmaceutically acceptable salt thereof, or a pharmaceutical composition of any of the foregoing may be administered to treat gastrointestinal bleeding such as upper gastrointestinal bleeding, ulcerative colitis, or hemorrhagic gastritis.

In certain embodiments, a compound of Formula (I), a pharmaceutically acceptable salt thereof, or a pharmaceutical composition of any of the foregoing may be administered to treat diffuse bleeding such uterine bleeding.

In certain embodiments, a compound of Formula (I), a pharmaceutically acceptable salt thereof, or a pharmaceutical composition of any of the foregoing may be administered to treat bleeding associated with child birth including post partum hemorrhage.

In certain embodiments, a compound of Formula (I), a pharmaceutically acceptable salt thereof, or a pharmaceutical composition of any of the foregoing may be administered to treat intracavitary bleeding (bleeding that occurs in organs) such as bleeding in the brain, inner ear, or eyes.

In certain embodiments, a compound of Formula (I), a pharmaceutically acceptable salt thereof, or a pharmaceutical composition of any of the foregoing may be administered to treat bleeding in organs and tissue where there is limited ability to apply mechanical or surgical hemostasis.

In certain embodiments, a compound of Formula (I), a pharmaceutically acceptable salt thereof, or a pharmaceutical composition of any of the foregoing may be administered to treat bleeding associated with surgery or trauma in subjects having acute hemarthroses (bleedings in joints), chronic hemophilic arthropathy, hematomas, (e.g., muscular, retroperitoneal, sublingual and retropharyngeal), bleedings in other tissue, hematuria (bleeding from the renal tract), renal hemorrhage, nasal hemorrhage, pulmonary hemorrhage, cerebral hemorrhage, intracerebral hemorrhage, subarachnoid hemorrhage, subdural hemorrhage, epidural hemorrhage, surgery (e.g., hepatectomy), dental extraction, and gastrointestinal bleedings.

Compounds of Formula (I), pharmaceutically acceptable salts thereof, and pharmaceutical compositions of any of the foregoing may be used to treat drug-induced bleeding. For example, bleeding may occur in subjects on anticoagulant therapy in whom a defective hemostasis has been induced by the therapy given. Anticoagulant therapy can be given to prevent thromboembolic disease and can include administration of heparin, other forms of proteoglycans, warfarin or other forms of vitamin K-antagonists as well as aspirin and other platelet aggregation inhibitors, such as, for example, antibodies or other inhibitors of GP IIb/IIIa activity. Bleeding may also be due to thrombolytic therapy which involves combined treatment with an antiplatelet agent (e.g., acetylsalicylic acid), an anticoagulant (e.g., heparin), and a fibrinolytic agent (e.g., tissue plasminogen activator, tPA).

In certain embodiments, a compound of Formula (I), a pharmaceutically acceptable salt thereof, or a pharmaceutical composition of any of the foregoing may be administered to increase ultrafiltration capacity in dialysis (Kuriyama et al., Peritoneal Dialysis International 1999, 19(1), 38-44).

In certain embodiments, a compound of Formula (I), a pharmaceutically acceptable salt thereof, or a pharmaceutical composition of any of the foregoing may be administered to treat bleeding during and after biopsy including, for example, liver biopsy, kidney biopsy, lung biopsy, tumor biopsy, gastrointestinal biopsy, and cervical conization.

In certain embodiments, a compound of Formula (I), a pharmaceutically acceptable salt thereof, or a pharmaceutical composition of any of the foregoing may be administered to restore and/or promote hemostasis in a patient. Hemostasis refers to the physiologic process whereby bleeding is halted. Hemostatic agents are those that prevent, treat or ameliorate abnormal bleeding, such as abnormal bleeding cause by a bleeding disorder or bleeding episode. Disorders of hemostasis include, for example, platelet disorders, such as idiopathic thrombocytopenic purpura, and disorders of coagulation such as hemophilia. Hemostasis can also refer to the complex interaction between vessels, platelets, coagulation factors, coagulation inhibitors and fibrinolytic proteins to maintain the blood within the vascular compartment in a fluid state. The objective of the hemostatic system is to preserve intravascular integrity by achieving a balance between hemorrhage and thrombosis. Promoting hemostasis refers to the process of contributing to or improving hemostasis in a subject. For example, an agent that promotes hemostasis can be an agent that reduces abnormal bleeding, such as by halting bleeding more rapidly, or by reducing the amount of blood loss.

In certain embodiments, a compound of Formula (I), a pharmaceutically acceptable salt thereof, or a pharmaceutical composition of any of the foregoing may be administered to treat bleeding associated with burst arteries and/or ruptured aneurysms in a patient.

When used to treat or prevent bleeding in a patient a therapeutically effective amount of one or more compounds of Formula (I) can be administered or applied singly, or in combination with other agents including other antifibrinolytic agents. When used to treat or prevent bleeding associated with brain surgery, a therapeutically effective amount of one or more compounds of Formula (I) can be administered or applied singly, or in combination with a calcium channel blocker. The therapeutically effective amount of one or more compounds of Formula (I) can also deliver a compound of Formula (I) in combination with another pharmaceutically active agent, including another compound of Formula (I).

Dosage forms, upon releasing a tranexamic acid prodrug of Formula (I), can provide tranexamic acid upon in vivo administration to a patient. The promoiety or promoieties of the prodrug of Formula (I) can be cleaved either chemically and/or enzymatically. One or more enzymes present in the stomach, intestinal lumen, intestinal tissue, blood, liver, brain or any other suitable tissue of a mammal can enzymatically cleave the promoiety or promoieties of the prodrug. If the promoiety or promoieties are cleaved after absorption by the gastrointestinal tract, tranexamic acid prodrugs of Formula (I) can be absorbed into the systemic circulation from the large intestine. In certain embodiments, the promoiety or promoieties are cleaved after absorption by the gastrointestinal tract. In certain embodiments, the promoiety or promoieties are cleaved in the gastrointestinal tract and tranexamic acid is absorbed into the systemic circulation form the large intestine. In certain embodiments, the tranexamic acid prodrug is absorbed into the systemic circulation from the gastrointestinal tract, and the promoiety or promoieties are cleaved in the systemic circulation, after absorption of the tranexamic acid prodrug from the gastrointestinal tract.

The amount of tranexamic acid prodrug that will be effective in the treatment of a particular disorder or condition disclosed herein can depend on the nature of the disorder or condition, and can be determined by standard clinical techniques known in the art. In addition, in vitro or in vivo assays can optionally be employed to help identify optimal dosage ranges. The amount of a compound administered can depend on, among other factors, the subject being treated, the weight of the subject, the severity of the affliction, the manner of administration, and the judgment of the prescribing physician.

In certain embodiments, a dosage form are adapted to be administered to a patient no more than twice per day, and in certain embodiments, only once per day. Dosing can be provided alone or in combination with other drugs and can continue as long as required for effective treatment.

Suitable daily dosage ranges for oral administration can range from about 2 mg to about 50 mg of tranexamic acid equivalents per kilogram body weight.

In certain embodiments, a compound of Formula (I) may be administered to treat bleeding in a patient in an amount from about 50 mg-equivalents to about 2,000 mg-equivalents tranexamic acid per day, from about 100 mg-equivalents to about 1,500 mg-equivalents tranexamic acid per day, from about 200 mg-equivalents to about 1,000 mg-equivalents tranexamic acid per day, or in any other appropriate daily dose.

In certain embodiments, a compound of Formula (I) may be administered to treat bleeding in a patient so as to provide a therapeutically effective concentration of tranexamic acid in the blood or plasma of the patient. In certain embodiments, a therapeutically effective concentration of tranexamic acid in the blood or plasma of a patient is from about 1 μg/mL to about 60 μg/mL, from about 2 μg/mL to about 50 μg/mL, from about 5 μg/mL to about 40 μg/mL, from about 5 μg/mL to about 20 μg/mL, and in certain embodiments from about 5 μg/mL to about 10 μg/mL. In certain embodiments, a therapeutically effective concentration of tranexamic acid in the blood or plasma of a patient is at least about 2 μg/mL, at least about 5 μg/mL, at least about 10 μg/mL, at least about 15 μg/mL, at least about 25 mg/mL, and in certain embodiments at least about 30 μg/mL. In certain embodiments, a therapeutically effective concentration of tranexamic acid in the blood or plasma of a patient is less than amount that causes unacceptable adverse effects including adverse effects to homeostasis. In certain embodiments, a therapeutically effective concentration of tranexamic acid in the blood or plasma of a patient is an amount sufficient to restore and/or maintain homeostasis in the patient.

In certain embodiments, a compound of Formula (I) may be administered to treating bleeding in a patient so as to provide a therapeutically effective concentration of tranexamic acid in the blood or plasma of a patient for an extended period of time such as, for example, for at least about 4 hours, for at least about 6 hours, for at least about 8 hours, for at least about 10 hours, and in certain embodiments, for at least about 12 hours.

The amount administered may vary depending upon whether the compound of Formula (I) is administered prophylactically prior to bleeding, during a bleeding episode, and/or following a bleeding episode. The amount administered may vary during a treatment regimen.

A compound of Formula (I), a pharmaceutically acceptable salt thereof, or a pharmaceutical composition of any of the foregoing may be included in a kit that may be used to administer the compound to a patient for treating bleeding. A kit can include a pharmaceutical composition comprising a compound of Formula (I) suitable for administration to a patient and instructions for oral administering the pharmaceutical composition to a patient. A kit can include one or more containers for containing one or more pharmaceutical compositions and may include divided containers such as a divided bottle or a divided foil packet. A container can be any appropriate shape or form which is made of a pharmaceutically acceptable material. A particular container can depend on the dosage form and the number of dosage forms provided. Instructions provided with a kit can include directions for administration and may include a memory aid. Instructions supplied with a kit may be printed and/or supplied, for example, as an electronic-readable medium, a video cassette, an audiotape, a flash memory device, or may be published on an internet web site or distributed to a patient as an electronic mail. A memory aid may be a written memory aid, which contains information and/or instructions for the physician, pharmacist, and/or patient to facilitate compliance with a dosing regimen. A memory aid may also be mechanical or electronic. When a therapeutic regimen includes administration of a compound of Formula (I) and at least on other therapeutic agent, a kit can include the at least one other therapeutic agent in the same or separate container as the compound of Formula (I), respectively.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A method of treating bleeding caused by a wound in a subject, comprising orally administering to the subject a therapeutically effective amount of a tranexamic acid prodrug at least about 1 hour prior to incurring the wound.

2. The method of claim 1, wherein the tranexamic acid prodrug is a compound of Formula (I): 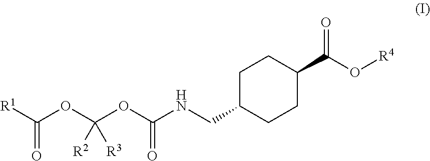
or a pharmaceutically acceptable salt thereof, wherein:
R¹ is selected from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently selected from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and
R⁴ is selected from hydrogen, alkyl, substituted alkyl, aryl, substituted aryl, arylalkyl, substituted arylalkyl, aryldialkylsilyl, substituted aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, trialkylsilyl, and substituted trialkylsilyl.

3. The method of claim 2, wherein:
R¹ is selected from isopropyl, isobutyl, and phenyl;
R² is hydrogen;
R³ is selected from methyl and isopropyl; and
R⁴ is hydrogen.

4. The method of claim 2, wherein:
R¹ is isopropyl;
one of R² and R³ is hydrogen; and
the other of R² and R³ is methyl; and R⁴ is hydrogen.

5. The method of claim 2, wherein the compound is selected from:
(+)-trans-4-({[(1S)-1-(2-methylpropanoyloxy)ethoxy]carbonylamino}methyl)-cyclohexanecarboxylic acid;
(−)-trans-4-({[(1R)-1-(2-methylpropanoyloxy)ethoxy]carbonylamino}methyl)-cyclohexanecarboxylic acid;
a pharmaceutically acceptable salt of any of the foregoing; and
mixtures of any of the foregoing.

6. The method of claim 2, wherein the subject has a bleeding disorder.

7. The method of claim 2, comprising orally administering the tranexamic acid prodrug to the subject after the wound is incurred.

8. The method of claim 2, wherein the wound is incurred by a surgery.

9. The method of claim 8, wherein the surgery comprises a biopsy.

10. The method of claim 9, wherein the biopsy is chosen from kidney biopsy and liver biopsy.

11. The method of claim 8, wherein the surgery is chosen from brain surgery, orthopedic surgery, cardiovascular surgery, gynecological surgery, liver transplant, and eye surgery.

12. The method of claim 2, wherein the wound is incurred in combat.

13. The method of claim 2, wherein the wound is the result of trauma.

14. The method of claim 2, wherein the wound is intracavitary.

15. The method of claim 2, wherein the tranexamic acid prodrug is administered as a sustained release oral formulation.

16. A method of treating gastrointestinal bleeding in a subject, comprising orally administering to the subject a therapeutically effective amount of a tranexamic acid prodrug.

17. A method of treating drug-induced bleeding in a subject, comprising orally administering to the subject a therapeutically effective amount of a tranexamic acid prodrug.
